(19) United States
(12) Patent Application Publication
(10) Pub. No.: US 2026/0315340 A1
(43) Pub. Date: Oct. 15, 2026

(54) ORAL COMPOSITION

(71) Applicant: LG H&H Co., LTD., Seoul (KR)

(72) Inventors: Jung-Heon YU, Seoul (KR); Kyoung-Hee OH, Seoul (KR)

(73) Assignee: LG H&H Co., LTD., Seoul (KR)

(21) Appl. No.: 19/643,078

(22) Filed: Apr. 9, 2026

(30) Foreign Application Priority Data
Apr. 10, 2025 (KR) 10-2025-0047079

(51) Int. Cl. A61K 8/19; A61K 8/20; A61K 8/24; A61Q 11/00

(52) U.S. Cl. CPC A61K 8/19; A61K 8/20; A61K 8/24; A61Q 11/00; A61K 2800/522; A61K 2800/92

(57) ABSTRACT

The present disclosure relates to an oral composition, and specifically relates to an oral composition having improved physical stability, irritability, and sensory quality of a product. The oral composition including iron oxide and an inorganic salt in combination according to the present disclosure can significantly improve the problem of discoloration of iron oxide with time, causes significantly low irritation on oral mucosa and shows significantly high sensory quality, such as taste and air bubbles.

### TECHNICAL FIELD

The present disclosure relates to an oral composition, and specifically relates to an oral composition having improved physical stability, irritability and sensory quality of a product.

### BACKGROUND ART

Recently, consumers put importance not only on oral health but also on the appearance, physical stability, and sensory satisfaction in use of a product. The stability of color in an oral composition is one of the important factors for judging consumer reliability and product quality. In general, an oral composition, such as toothpaste, may contain not only functional ingredients but also a colorant in consideration of consumer preferences. As negative perceptions of tar pigments spread, inorganic colorants, such as iron oxide, have been widely used to realize red or yellow colors in oral products, such as toothpaste.

However, conventional iron oxide-prescribed toothpaste formulations have a possibility of discoloration (decoloring) during long-term storage. Such a discoloration phenomenon not only lowers the appearance quality of a product but also may act as a factor that lowers consumer reliability and satisfaction.

In addition, prescriptions containing salt are sometimes used to improve the sensory quality of an oral composition. Salt functions to improve oral cleaning power, to control cell osmotic pressure and to supply minerals, and at a certain concentration, it increases a feeling of air bubbles in the oral cavity and provides a refreshing feeling of use. However, if the salt concentration is excessive, problems may occur in that irritation increases, an unpleasant salty taste is emphasized, and the feeling of air bubbles decreases. Such problems deteriorate the overall sensory quality of the product and may be a cause of lowering consumer satisfaction during long-term use.

### DISCLOSURE

### Technical Problem

A problem to be solved by the present disclosure is to solve the problem of discoloration (decoloring) with time by providing an oral composition including iron oxide and a salt in combination. In addition, another problem to be solved by the present disclosure is to improve the quality of properties of a product while not detracting from the sensory quality by incorporating an appropriate concentration of salt to the oral composition.

### Technical Solution

The inventors of the present disclosure have conducted intensive studies to solve the problem that red iron oxide-prescribed toothpaste according to the prior art is discolored (decolored) with time to cause deterioration of the appearance quality of a product. In one aspect of the present disclosure, the inventors have found that when such red iron oxide-prescribed toothpaste is applied to a container made of a gas barrier material, the problem of discoloration (decoloring) with time is significantly increased, and an attempt was made to develop an oral composition that can effectively solve the above-mentioned problem.

The present disclosure provides an oral composition for preventing discoloration of iron oxide with time, the oral composition including iron oxide and an inorganic salt. The oral composition can highly effectively prevent discoloration of iron oxide with time by including iron oxide in combination with an inorganic salt.

According to an embodiment of the present disclosure, the iron oxide may include at least one selected from the group consisting of red iron oxide, yellow iron oxide, black iron oxide and brown iron oxide, but is not limited thereto. Preferably, the iron oxide may be red iron oxide, and the oral composition according to the present disclosure can significantly reduce the problem of discoloration of red iron oxide with time.

According to an embodiment of the present disclosure, the inorganic salt may include at least one selected from the group consisting of sodium chloride, potassium chloride, potassium nitrate, sodium bicarbonate, sodium carbonate, sodium dihydrogen phosphate, calcium hydroxide, potassium pyrophosphate, sodium pyrophosphate, sodium phosphate monobasic, sodium phosphate tribasic, disodium succinate, sodium citrate, sodium polyphosphate, potassium phosphate monobasic, and potassium phosphate dibasic, but is not limited thereto. Preferably, the inorganic salt may be sodium chloride, sodium bicarbonate or potassium pyrophosphate, and more preferably sodium chloride, through which the oral composition according to the present disclosure can significantly reduce the problem of discoloration of iron oxide with time.

According to an embodiment of the present disclosure, the iron oxide may be contained in an amount of 0.0001 wt % to 1 wt %, preferably 0.001 wt % to 0.1 wt %, based on the total weight of the oral composition. When the content of iron oxide is larger than 1 wt % based on the total weight of the oral composition, the sensory quality, such as taste or air bubbles, may be degraded. When the content of iron oxide is less than 0.0001 wt %, a desired coloring effect may not be realized sufficiently.

According to an embodiment of the present disclosure, the inorganic salt may be contained in an amount of 0.1 wt % to 8 wt %, preferably 0.3 wt % to 5 wt %, based on the total weight of the oral composition. When the content of inorganic salt is larger than 8 wt % based on the total weight of the oral composition, an irritative feeling and an unpleasant taste may increase and a feeling of air bubbles may be decreased, thereby causing deterioration of sensory quality. When the content of inorganic salt is less than 0.1 wt %, the problem of discoloration of iron oxide with time may not be solved.

According to an embodiment of the present disclosure, the weight ratio (iron oxide:inorganic salt) of iron oxide and inorganic salt may be 1:20 to 1:1800, preferably 1:40 to 1:1400, and more preferably 1:80 to 1:1000. When the iron oxide and inorganic salt are contained within the above-defined range of weight ratio, the problem of discoloration of iron oxide with time can be significantly reduced, and the irritability, taste and air bubbling degree may be improved significantly.

The present disclosure also provides an oral composition for a gas barrier container, including iron oxide and an inorganic salt. The oral composition for a gas barrier container can significantly improve the problem of discoloration of iron oxide with time in the gas barrier container through the combination of iron oxide and the inorganic salt.

According to an embodiment of the present disclosure, the gas barrier container may be used to prevent oxidation and discoloration of an oral composition (e.g. toothpaste, etc.) caused by introduction of oxygen, to prevent a change in viscosity of contents and degradation of quality, caused by moisture evaporation, and to prevent a decrease in aroma and active ingredients caused by a loss of volatile ingredients. The material forming the gas barrier container may be at least one selected from the group consisting of: a plastic-based material, such as ethylene vinyl alcohol, polyvinylidene chloride, polyethylene terephthalate and polyethylene naphthalate; a metal-based material including aluminum and an aluminum barrier laminate (ABL) tube, a metal deposited film, etc.: and a multilayer composite material, such as a plastic barrier laminate (PBL) tube, but is not limited thereto. Preferably, the gas barrier container may be a tube made of ABL, and when the oral composition is introduced thereto, the problem of discoloration of iron oxide with time can be significantly improved.

In addition, the present disclosure provides an oral product including: an oral composition including iron oxide and an inorganic salt; and a gas barrier container supporting the oral composition. According to an embodiment of the present disclosure, the oral product may include various types of compositions and tools used to maintain hygiene in teeth, gums and oral cavities and to prevent oral diseases. Such products have the purpose of preventing tooth decay, removing plaque and tartar, removing bad breath or maintaining oral health and may be classified into various types depending on the use and shape.

According to an embodiment of the present disclosure, the oral product may include a composition applied to teeth and oral mucosa, and may be toothpaste, an oral cleaner, an oral spray, an oral gel, an oral powder or an oral whitening product, but is not limited thereto. In addition, the oral product according to the present disclosure may be in the form of liquid, solid, suspension, gel or aerosol, but is not limited thereto.

According to an embodiment of the present disclosure, the oral composition may further include conventional ingredients, such as a wetting agent, a polishing agent, a medicinal agent, a sweetening agent, a pH modifier, a preservative, a binder, a colorant, a flavoring, a bubbling agent, or the like, depending on the formulation and purpose of use. With regard to the wetting agent, ingredients selected from the group consisting of glycerin concentrate, glycerin, aqueous sorbitol solution, aqueous amorphous sorbitol solution, polyethylene glycols and propylene glycol may be used alone or in combination. In addition, with regard to the polishing agent, any one selected from the group consisting of precipitated silica, silica gel, zirconium silicate, calcium monohydrogen phosphate, anhydrous calcium monohydrogen phosphate, hydrous alumina, light calcium carbonate, heavy calcium carbonate, calcium pyrophosphate, insoluble metaphosphate and aluminum silicate may be used. Further, with regard to the medicinal agent, sodium fluoride, sodium monofluorophosphate, tin fluoride, chlorhexidine, allantoin chlorohydroxyaluminate, aminocaproic acid, zinc chloride, pyridoxine hydrochloride, tocopherol acetate, enzymes, etc. may be used alone or in combination. Additionally, with regard to the binder, sodium carboxymethyl cellulose, carbomer, carrageenan, xanthan gum, alginates, etc. may be used. In addition, with regard to the bubbling agent, an anionic surfactant, such as sodium alkyl sulfate or sodium lauryl sulfate, and a non-ionic surfactant, such as sucrose fatty acid ester, sorbitan fatty acid ester, polyoxyethylene polyoxypropylene copolymer (poloxamer), polyoxyethylene hydrogenated castor oil or polyoxyethylene sorbitan fatty acid ester may be used alone or in combination. In addition, as the sweetening agent, saccharin, xylitol, erythritol, aspartame, etc. may be used. In addition, as the pH modifier, sodium phosphate, disodium hydrogen phosphate, citric acid, triethanol amine (TEA), etc. may be used. In addition, with regard to the preservative, benzoic acid, methyl paraben, propyl paraben, sodium benzoate, etc. may be used. With regard to the flavoring, peppermint oil, spearmint oil, menthol, etc. may be used in combination. Further, with regard to other additives, enzymes, such as dextranase, may be used.

All ingredients described in the present disclosure preferably do not exceed the maximum use level stipulated in the relevant laws and regulations of Korea, China, the United States, Europe, Japan, etc. (for example, regulations on cosmetic safety standards, etc. (Korea) and cosmetic safety technology norms (China)). In other words, preferably, the oral composition according to the present disclosure includes the ingredients according to the present disclosure to a content limit allowed by the relevant laws and regulations of each country.

### Advantageous Effects

The oral composition according to the present disclosure can significantly improve the problem of discoloration of iron oxide with time, causes significantly low irritation on oral mucosa and shows significantly high sensory quality, such as taste and air bubbles.

### MODE FOR INVENTION

Hereinafter, specific embodiments will be described in detail to help understanding of the present disclosure. However, the embodiments according to the present disclosure may be modified in various different forms, and the scope of the present disclosure should not be construed as being limited to the following embodiments. The embodiments of the present disclosure are provided to more completely explain the present disclosure to a person having average knowledge in the field to which the present invention belongs.

### PREPARATION OF EXAMPLES AND COMPARATIVE EXAMPLES

The toothpaste compositions according to Examples 1 to 5 and Comparative Examples 1 to 4 were prepared according to the compositions as shown in the following Table 1 by using a conventional method.

TABLE 1

| | Examples | | | | | Comparative Examples | | | |
|---|---|---|---|---|---|---|---|---|---|
| (Unit: %) | 1 | 2 | 3 | 4 | 5 | 1 | 2 | 3 | 4 |
| Sorbitol solution (70%) | 60 | 60 | 60 | 60 | 60 | 60 | 60 | 60 | 60 |
| Dental type silica | 15 | 15 | 15 | 15 | 15 | 15 | 15 | 15 | 15 |
| Sodium lauryl sulfate | 2 | 2 | 2 | 2 | 2 | 2 | 2 | 2 | 2 |
| Sodium saccharin | 1 | 1 | 1 | 1 | 1 | 1 | 1 | 1 | 1 |
| Sodium carboxymethyl | 1 | 1 | 1 | 1 | 1 | 1 | 1 | 1 | 1 |
| cellulose | | | | | | | | | |
| Sodium fluoride | 0.22 | 0.22 | 0.22 | 0.22 | 0.22 | 0.22 | 0.22 | 0.22 | 0.22 |
| Red iron oxide | 0.005 | 0.005 | 0.005 | 0.005 | | 0.005 | 0.005 | 0.005 | |
| Yellow iron oxide | | | | | 0.005 | | | | 0.005 |
| Sodium chloride | 0.5 | 2 | | | 2 | 0.05 | 10 | | |
| Sodium bicarbonate | | | 2 | | | | | | |
| Potassium pyrophosphate | | | | 2 | | | | | |
| Flavoring | 1 | 1 | 1 | 1 | 1 | 1 | 1 | 1 | 1 |
| Purified water | q.s. | q.s. | q.s. | q.s. | q.s. | q.s. | q.s. | q.s. | q.s. |

### Test Example 1: Evaluation of Physical Stability with Time

Each of the toothpaste compositions according to Examples 1 to 5 and Comparative Examples 1 to 4 was introduced into a tube made of aluminum barrier laminate (ABL) and stored therein at 50° C. for 1 month to evaluate the physical stability of the toothpaste composition with time.

TABLE 2

| | Physical stability |
|---|---|
| Example 1 | Good |
| Example 2 | Good |
| Example 3 | Good |
| Example 4 | Good |
| Example 5 | Good |
| Comparative Example 1 | Decolored |
| Comparative Example 2 | Good |
| Comparative Example 3 | Decolored |
| Comparative Example 4 | Decolored |

As can be seen from the results of Table 2, Comparative Example 1 containing sodium chloride in an amount (0.05 wt %) of less than 0.1 wt %, or Comparative Examples 3 and 4 including no sodium chloride cause a problem of decoloring of iron oxide with time.

### Test Example 2: Evaluation of Irritability to Oral Mucosa

The toothpaste prepared according to each of Examples and Comparative Examples was distributed to 15 subjects sensitive to mucosal irritation, and then evaluation of irritation to oral mucosa was carried out with a 4-point scale. Approximately 1 g of the toothpaste composition was squeezed into a toothbrush and evaluated in terms of oral mucosa irritation when brushing for 3 minutes.

### Measure of Evaluation

1 point: Feel no irritation
2 point: Feel almost no irritation
3 point: Feel irritation
4 point: Feel strong irritation

TABLE 3

| | Oral mucosa irritability |
|---|---|
| Example 1 | 1.2 |
| Example 2 | 1.4 |
| Example 3 | 1.3 |
| Example 4 | 1.4 |
| Example 5 | 1.4 |
| Comparative Example 1 | 1.3 |
| Comparative Example 2 | 3.8 |
| Comparative Example 3 | 1.2 |
| Comparative Example 4 | 1.3 |

As can be seen from the results of Table 3, Comparative Example 2 including sodium chloride in an amount (10 wt %) of larger than 8 wt % was confirmed to cause strong irritation to oral mucosa.

### Test Example 3: Evaluation of Sensory Quality of Toothpaste

The toothpaste prepared according to each of Examples and Comparative Examples was distributed to 15 consumers, and then evaluation of unpleasant taste during and after brushing was conducted with a 5-point scale. Approximately 1 g of the toothpaste composition was squeezed into a toothbrush and evaluated in terms of unpleasant taste during and after brushing when brushing for 3 minutes.

### Sensory Satisfaction

1. How satisfied are you with the taste of the toothpaste you used this time?
1) very dissatisfied; 2) dissatisfied; 3) neutral; 4) satisfied; 5) very satisfied
2. What is the degree of unpleasant taste (bitterness, sourness, etc.) during brushing of the toothpaste you used this time?
1) very weak, 2) weak, 3) moderate, 4) strong, 5) very strong
3. What is the degree of unpleasant taste (bitterness, sourness, etc.) after brushing of the toothpaste you used this time?
1) very weak, 2) weak, 3) moderate, 4) strong, 5) very strong

TABLE 4

| | Sensory | Unpleasant | Unpleasant |
|---|---|---|---|
| | quality | taste | taste |
| | satisfaction | during use | after use |
| Example 1 | 4.4 | 1.4 | 1.3 |
| Example 2 | 4.5 | 1.4 | 1.3 |
| Example 3 | 4.5 | 1.5 | 1.4 |
| Example 4 | 4.5 | 1.4 | 1.3 |
| Example 5 | 4.4 | 1.4 | 1.4 |
| Comparative Example 1 | 4.4 | 1.2 | 1.2 |
| Comparative Example 2 | 1.5 | 4.5 | 4.4 |
| Comparative Example 3 | 4.6 | 1.2 | 1.3 |
| Comparative Example 4 | 4.6 | 1.4 | 1.4 |

As can be seen from the results of Table 4, Comparative Example 2 including sodium chloride in an amount (10 wt %) of larger than 8 wt % shows an increase in unpleasant taste and causes significant degradation of sensory quality satisfaction.

### Test Example 4: Measurement of Air Bubbling Degree of Toothpaste

Each toothpaste was applied to a tooth-shaped jig and brushing for 3 minutes was carried out 200 times while flowing saliva or purified water at a rate of 1 mL per minute, the air bubbles generated after brushing was received with a mess-cylinder, and the amount thereof was measured, by using a TP foam tester instrument.

TABLE 5

| | Air bubbling degree (mL) |
|---|---|
| Example 1 | 52 |
| Example 2 | 50 |
| Example 3 | 49 |
| Example 4 | 51 |
| Example 5 | 49 |
| Comparative Example 1 | 51 |
| Comparative Example 2 | 24 |
| Comparative Example 3 | 52 |
| Comparative Example 4 | 51 |

As can be seen from the results of Table 5, Comparative Example 2 including sodium chloride in an amount (10 wt %) of larger than 8 wt % causes a significant decrease in air bubbling degree.

It can be seen from Test Examples 1 to 4 that when iron oxide and an inorganic salt are formulated in combination in a specific content range, a product using iron oxide shows improved physical stability while reducing oral mucosa irritation and providing sensory quality, such as taste and air bubbles, significantly.

1. An oral composition for preventing discoloration of iron oxide with time, comprising iron oxide and an inorganic salt.

2. The oral composition for preventing discoloration of iron oxide with time according to claim 1, wherein the iron oxide comprises at least one selected from the group consisting of red iron oxide, yellow iron oxide, black iron oxide, and brown iron oxide.

3. The oral composition for preventing discoloration of iron oxide with time according to claim 1, wherein the inorganic salt comprises at least one selected from the group consisting of sodium chloride, potassium chloride, potassium nitrate, sodium bicarbonate, sodium carbonate, sodium dihydrogen phosphate, calcium hydroxide, potassium pyrophosphate, sodium pyrophosphate, sodium phosphate monobasic, sodium phosphate tribasic, disodium succinate, sodium citrate, sodium polyphosphate, potassium phosphate monobasic, and potassium phosphate dibasic.

4. The oral composition for preventing discoloration of iron oxide with time according to claim 1, wherein the iron oxide is contained in an amount of 0.0001 wt % to 1 wt % based on the total weight of the oral composition.

5. The oral composition for preventing discoloration of iron oxide with time according to claim 1, wherein the inorganic salt is contained in an amount of 0.1 wt % to 8 wt % based on the total weight of the oral composition.

6. The oral composition for preventing discoloration of iron oxide with time according to claim 1, wherein the weight ratio (iron oxide:inorganic salt) of iron oxide and inorganic salt is 1:20 to 1:1800.

7. An oral composition for a gas barrier container, comprising iron oxide and an inorganic salt.

8. An oral product comprising: an oral composition comprising iron oxide and an inorganic salt; and a gas barrier container supporting the oral composition.
